(19) United States
(12) Patent Application Publication
(10) Pub. No.: US 2007/0142844 A1
(43) Pub. Date: Jun. 21, 2007

(54) Cervical seal apparatus and method of sealing a cervix

(76) Inventors: Robert Kotmel, Burlingame, CA (US); Russel M. Sampson, Palo Alto, CA (US); Tay Doan, Santa Clara, CA (US); Nadia Matov, San Jose, CA (US); Estella H. Hilario, Los Altos, CA (US)

Correspondence Address: FISH & RICHARDSON P.C., PO BOX 1022, MINNEAPOLIS, MN, 55440-1022, US

(21) Appl. No.: 11/313,446

(22) Filed: Dec. 20, 2005

(51) Int. Cl. A61B 17/42

(52) U.S. Cl. 606/119

(57) ABSTRACT

Techniques and apparatus for providing a compressive force to a cervix to provide a seal is described. For example, a seal can be provided between the cervix and a medical device extending therein. In one implementation, clamping tips positioned at the end of movable arms are provided to clamp the cervix and provide a seal. In another implementation, an adjustable strap is provided at the end of an elongate member to strap around a cervix and provide a seal.

### TECHNICAL FIELD

This invention relates to medical devices.

### BACKGROUND

Perimenopausal women frequently encounter irregular uterine bleeding, and often present with inter-menstrual spotting or heavy periods. This irregularity is called menorrhagia. Women suffering from menorrhagia experience fatigue, anemia, embarrassing accidents, and restricted activity, and may undergo surgical and medical treatment. Available treatment includes hormone therapy, endometrial biopsy with or without dilation and curettage, endometrial ablation, and hysterectomy.

One treatment using endometrial ablation for treating menorrhagia includes NovaSure Endometrial Ablation (NEA). NEA is one example of an endometrial ablation procedure using a global endometrial ablation device for performing ablation via controlled vaporization of the endometrium, a layer of cells that contributes to irregular uterine bleeding. A cervix is dilated, and a mesh triangular array is delivered via a slender, handheld device and inserted into a uterus of a patient. The shape of the mesh triangular array is configured to generally resemble a profile of a uterine cavity.

Prior to energizing the array, a surgical technique is applied to bring the uterine cavity into close contact with the mesh triangular array. After electrical energy (e.g., radio frequency) has been delivered to the endometrial lining of the uterine cavity via the array for one to two minutes to destroy the endometrium, the lining is removed, the mesh triangular array is retracted and the handheld device is removed from the uterus.

Inserting the handheld device into the uterine cavity is typically a blind procedure. A physician inserts the handheld device transcervically, in line with the axis of the uterus, and advances the device until it reaches the uterine cavity. Exerting in excess of a threshold force on the handheld device during insertion can lead to perforation of the uterine wall by the device. The risk of injuring bowels and other vital organs increases if the uterine wall perforates at the time electrical energy is applied. To mitigate such risk, prior to applying electrical energy to the uterine cavity (surface of the endometrium), a cavity integrity assessment test is performed before conducting the endometrial ablation procedure.

During the cavity integrity assessment test, carbon dioxide CO₂ is delivered to the uterine cavity by the handheld device to pressurize the entire uterus to a known pressure. Based on whether the pressure in the uterine cavity is maintained, it can be determined if a leak exists in the uterine cavity. However, it is often difficult to discern the difference between a leak caused by perforation of the uterine wall or that by a loose seal between the cervix and an outer shaft/cervical collar of the handheld device.

### SUMMARY

This invention relates to medical devices and techniques. In general, in one aspect, the invention features a cervical seal device including an elongate member and a strap and a method for using the device to provide a compressive seal to a cervix. The elongate member has a distal end and a proximal end, the distal end including an inner element coaxially positioned inside an outer element. The strap includes a first end and a second end, the first end connected to the inner element of the distal end of the elongate member and the second end connected to the outer element of the distal end of the elongate member. The strap forms a loop having a radius that is variable by rotating the inner element of the distal end of the elongate member to wind or unwind a length of the strap. The distal end of the elongate member is configured for insertion into a vaginal canal to an exterior of a cervix and the loop formed by the strap is configured to position around the exterior of the cervix and apply a compressive force to the cervix.

Implementations of the device can include one or more of the following features. The compressive force applied to the cervix can be varied by rotating the inner element of the distal end of the elongate member to wind the strap and decrease the radius of the loop formed by the strap. The distal end of the elongate member can include a detachable cartridge including the strap. The inner element of the distal end of the elongate member can be adapted to rotate substantially freely inside the outer element. An unwound length of the strap can be dependent upon a degree of rotation of the strap inside the inner element.

The elongate member can further include a rotary handle where the rotary handle is adapted to rotate the inner element to adjust a radius of the loop formed by the strap. The device can further include a locking mechanism to maintain the radius of the loop. The strap can be made of an elastic material selected from one of a group consisting of silicone, natural rubber, viton, latex, Buena-N, E-PM rubber, urethane, Santoprene, and polyurethane. In another implementation, the strap can be made of a non-elastic material selected from one of a group consisting of PVC mesh, polyester mesh or filaments coated with PVC, nylon mesh, metal, mylar, polyetheretherketone (PEEK), polyimide, polypropelyne and acrylonitrile butadiene styrene (ABS). The outer element at the distal end of the elongate member can be configured to vary the loop radius upon rotation of the outer element. The compressive force applied to the cervix can be substantially circumferential. The compressive force applied to the cervix can act over a portion of the cervix. The strap can include a plurality of frictional elements formed thereon, the frictional elements configured to preserve a strap position on the cervix and maintain the compressive force applied to the cervix.

In general, in another aspect, the invention features a cervical seal device including an outer sheath, and inner shaft, a flexible member and a handle, and a method for using the cervical seal device to apply a compressive force to a cervix. The outer sheath has a distal end and a proximal end and a lumen extending therebetween, the distal end including an aperture. The inner shaft is positioned within the lumen of the outer sheath, and a distal end of the inner shaft is connected to the flexible member. The flexible member has a first end and a second end. The first end is connected to the inner shaft and the second end is connected to the outer sheath or to the inner shaft. The flexible member forms a resizable loop extending from the aperture in the distal end of the outer sheath. The handle is attached to the proximal end of the outer sheath and the inner shaft. The handle includes at least a first component configured to be moved relative to a second component in a squeezing motion. Squeezing the handle retracts the inner shaft and at least the first end of the flexible member connected thereto within the outer sheath thereby reducing a size of the loop formed by the flexible member. The distal end of the outer sheath is configured for insertion into a vaginal canal to an exterior of a cervix and the loop formed by the flexible member is configured to position around the exterior of the cervix and apply a compressive force to the cervix.

In general, in another aspect, the invention features a cervical seal system including a clamping device and a strap, and a method for using the cervical seal system to apply a compressive force to a cervix. The clamping device includes two arms, each arm including a distal end and a proximal end with a handle positioned at the proximal end. The two arms are pivotally attached to one another at a point between the distal and proximal ends such that the arms are pivotable relative to each other. Moving the proximal ends toward each other moves the distal ends toward each other from an opened position into a closed position. A mating tip formed at the distal end of each arm is configured to mate with a capturing receptacle formed on the strap. The strap has at least two capturing receptacles mounted thereon, where each capturing receptacle is configured to receive a mating tip formed at the distal end of an arm. The strap thereby forms a loop configured to position around an exterior of a cervix and exert a compressive force on the cervix when the arms are moved into a substantially closed position.

Implementations of the device can include one or more of the following features. The strap can include more than two capturing receptacles to allow differing sized loops to be formed by the strap when mated with the mating tips. The device can further include a locking mechanism to maintain a radius of the loop formed by the strap. Each mating tip can be detachable from the corresponding arm. Each arm can be made of plastic materials selected from one of a group consisting of polyetheretherketone (PEEK), polysulfate, polypropelyne, acrylonitrile butadiene styrene (ABS) and ultem. Each mating tip can be made of plastic materials selected from one of a group consisting of polyetheretherketone (PEEK), polysulfate, polypropelyne, acrylonitrile butadiene styrene (ABS) and ultem. Each arm can include a supporting member operable to reduce a shaft deflection or bending stress. Each mating tip can be one of a ball tip, hemispherical tip, pan head, hook tip or a side pin. The strap can be made of an elastic material selected from one of a group consisting of silicone, natural rubber, viton, latex, Buena-N, E-PM rubber, urethane, Santoprene, and polyurethane. In another implementation, the strap can be made of a non-elastic material selected from one of a group consisting of PVC mesh, polyester mesh or filaments coated with PVC, nylon mesh, metal, mylar, polyetheretherketone (PEEK), polyimide, polypropelyne and acrylonitrile butadiene styrene (ABS).

In general, in one aspect, the invention features a method of providing a compressive force to a cervix. A first end of a strap is secured into a holding element formed at a distal end of an elongate member, where a second end of the strap is already secured to the distal end, the strap thereby forming an approximate loop. An elongate member is inserted through a vaginal canal into a proximity of the exterior of the cervix. A loop formed by the strap is positioned around the exterior of the cervix. A length of the strap is wound into the distal end of the elongate member to decrease a size of the loop and thereby exert a compressive force on the cervix.

Implementations of the method can include one or more of the following. Inserting the elongate member can include wrapping the strap externally around an existing tool positioned inside the vaginal canal externally without retracting the existing tool prior to positioning the loop around the exterior of the cervix. The second end of the strap can be secured to an inner element within the distal end of the elongate member and a holding element is attached to an outer element of the distal end, such that winding a length of the strap includes rotating a knob at a proximal end of the elongate member to rotate the inner element relative to the outer element. In another implementation, the second end of the strap can be secured to an inner element within the distal end of the elongate member and the holding element can be attached to an outer element of the distal end, such that winding a length of the strap includes squeezing a handle at a proximal end of the elongate member to rotate the inner element relative to the outer element.

In general, in another aspect, the invention features a method of providing a compressive force to a cervix. A distal end of an outer sheath having a distal end including an aperture, a proximal end, a lumen extending therebetween and an inner shaft positioned within the lumen, is inserted into a vaginal canal to the vicinity of a cervix. A flexible member is positioned around the cervix. The flexible member has a first end and a second end, where the first end is connected to the inner shaft and the second end is connected to the outer sheath or to the inner shaft. The flexible member forms a resizable loop extending from the aperture in the distal end of the outer sheath. A handle attached to the proximal end of the outer sheath and the inner shaft is squeezed. the handle including at least a first component configured to be moved relative to a second component in a squeezing motion, where squeezing the handle retracts the inner shaft and at least the first end of the flexible member connected thereto within the outer sheath thereby reducing a size of the loop formed by the flexible member and providing a compressive force to the cervix.

In general, in another aspect, the invention features a method of providing a compressive force to a cervix. A first end of a strap is secured onto a mating tip formed at a distal end of a first elongate arm and a second end of a strap is secured onto a mating tip formed at a distal end of a second elongate arm, where the first and the second elongate arms are pivotally connected to one another at a point of rotation between distal and proximal ends of the arms. The strap thereby forms an approximate loop. The arms and the strap are inserted through a vaginal canal into a proximity of the exterior of the cervix. The loop formed by the strap is positioned around the exterior of the cervix. The distal ends of the arms are moved toward one another to substantially close the loop formed by the strap and thereby exert a compressive force on the cervix.

Implementations of the method can include one or more of the following. The strap can include at least two capturing receptacles on the second end of the strap, and securing the second end of the strap onto a mating tip can include selecting a capturing receptacle on the second of the strap corresponding to a desired loop size.

In general, in another aspect, the invention features a medical device for providing a compressive force to a cervix. The device includes a substantially cylindrically shaped outer element, an inner shaft positioned with the outer element, and a strap including a first end and a second end, the first end connected to the inner element and the second end connected to the outer element. The strap forms a loop having a radius that is variable by rotating the inner element to wind or unwind a length of the strap. The outer element and inner shaft are configured to connect to an elongate member configured for insertion into a vaginal canal to an exterior of a cervix and the loop formed by the strap is configured to position around the exterior of the cervix and apply a compressive force to the cervix as the inner element is rotated by an adjustment means included on the elongate member to reduce the radius of the loop formed by the strap.

Implementations of the device can include one or more of the following. The strap can be made of an elastic material selected from one of a group consisting of silicone, natural rubber, viton, latex, Buena-N, E-PM rubber, urethane, Santoprene, and polyurethane. In another implementation, the strap can be made of a non-elastic material selected from one of a group consisting of PVC mesh, polyester mesh or filaments coated with PVC, nylon mesh, metal, mylar, polyetheretherketone (PEEK), polyimide, polypropelyne and acrylonitrile butadiene styrene (ABS).

In general, in another aspect, the invention features a cervical seal device including an elongate member, at least two elongate arms and at least two hemispherically shaped clamping tips. The elongate member has a distal end and a proximal end for engagement in a longitudinal direction into a vaginal canal. The each of the arms are pivotally mounted on the distal end of the elongate member for relative movement between an opened and a closed position. Each clamping tip is located on a distal end of each of the at least two arms, and is adapted to impart a compressive force on a cervix when the at least two arms are in a substantially closed position.

Implementations of the device can include one or more of the following features. The elongate member can further include an outer shaft and an inner shaft, where the at least two elongate arms are attached to a distal end of the inner shaft and are pivotally connected to a distal end of the outer shaft. A rotary handle can be mounted on the proximal end including a control knob. Rotating the control knob can move the inner shaft axially within the outer shaft switching the at least two elongate arms between the opened and closed positions. The device can include two handle grips, where each of the elongate arms is connected to a corresponding handle grip. Switching the at least two elongate arms between the opened and closed positions can be controlled based on a distance between the two handle grips. The device can include a locking mechanism adapted to maintain the elongate arms in a substantially closed position to maintain a compressive force on the cervix. The locking mechanism can be further configured to adjust separation and spacing between each clamping tip in discrete intervals. Each clamping tip included in the device can be substantially hemi-spherically shaped and can be detachable from the corresponding elongate arm. Each elongate arm can be made of plastic materials selected from one of a group consisting of polyetheretherketone (PEEK), polysulfate, polypropelyne, acrylonitrile butadiene styrene (ABS) and ultem. Each elongate arm can include a supporting member operable to reduce bending stress.

In one implementation the device includes four elongate arms, where each of the four elongate arms is pivotally mounted on the distal end of the elongate member for relative movement between an opened and a closed position. The device further includes four clamping tips, where each additional clamp tip is located on a distal end of a corresponding elongate arm.

In general, in another aspect, the invention features a cervical seal device including two elongate cross arms, grips and two hemispherically shaped clamping tips. Each of the arms includes a distal and a proximal end and they are pivotally connected to one another at a point between the distal and proximal ends. The grips are included on the proximal ends of the two arm and configured to move the distal ends of the arms between an opened and a closed position. Each clamping tip is located on the distal end of each arm and is adapted to impart a compressive force on a cervix when the arms are in a substantially closed position.

In one implementation, each clamping tip is pivotally attached to the distal end of each arm such that the clamping tip can swivel relative to the arm.

In general, in another aspect, the invention features a method of providing a compressive force to a cervix. A device including two elongate cross arms is inserted into a vaginal canal and into a proximity of the exterior of a cervix. Each of the arms includes a distal and a proximal end and are pivotally connected to one another at a point between the distal and proximal ends. Two hemispherically shaped clamping tips located at the distal ends of the arms are positioned about the exterior of the cervix. The proximal ends of the arms are moved toward one another to move the distal ends of the arms into a substantially closed position. The clamping tips thereby provide a compressive force on the cervix.

In general, in another aspect, the invention features a method of providing a compressive force to a cervix. A distal end of an elongate member including at least two elongate arms, where each of the arms is pivotally mounted on the distal end of the elongate member for relative movement between an opened and a closed position, is inserted into a vaginal canal into a proximity of the exterior of a cervix. At least two hemispherically shaped clamping tips are positioned at distal ends of the at least two elongate arms. The clamping tips are positioned about the exterior of the cervix. The arms are moved toward a substantially closed position, therefore exerting a compressive force on the cervix.

Implementations of the invention may include one or more of the following advantageous features. A physician can apply a compressive force to the cervix to provide a seal, which can be critical in certain procedures, such as performing the cavity integrity assessment test described above. In the implementation described using a strap, either retractable within a cartridge or loaded onto mating tips at the end of a handheld device, the strap length is adjustable to accommodate a wide range of cervix sizes. In the implementation described using clamping tips, a good mechanical advantage is provided, allowing the physician to squeeze the elongate arms together with one hand to gently, yet firmly, apply a compressive force to seal the cervix. A ratchet locking mechanism can permit a user to fasten the clamping tips onto the cervix, and release a hold thereon without compromising a compressive force exerted on the cervix by the cervical seal device. The cervical seal device can be manufactured such that a handle portion is reusable and a strap or clamping tip portion is disposable. An atraumatic tip design (e.g., hemi-spherical) can prevent scraping the cervical tissues (e.g., pinching or piercing) by edges of the elongate arms.

The cervical seal device can be used effectively without having to remove other devices that may already be inside the cervical canal and/or uterus. During a hysteroscopy procedure where the procedure is leaking a large amount of bodily fluid, fluids may leak within the cervical canal and around the hysteroscope tools positioned inside the cervical canal. The cervical seal device strap can be applied to the cervix to reduce or cease the fluid leaking without having to remove the hysteroscope tools.

The details of one or more embodiments of the invention are set forth in the accompanying drawings and the description below. Other features, objects, and advantages of the invention will be apparent from the description and drawings, and from the claims.

### DESCRIPTION OF DRAWINGS

The accompanying drawings, which are incorporated into and form a part of the specification, illustrate several aspects and embodiments of the present invention and, together with the general description given above and detailed description given below, serve to explain the principles of the invention. The drawings are only for the purpose of illustrating preferred embodiments of the invention and are not to be construed as limiting the invention. In the drawings:

FIGS. 1(a) and (b) show an embodiment of a cervical seal device.

FIG. 2(a) is a flowchart showing an exemplary process using the cervical seal device shown in FIGS. 1(a) and (b).

FIG. 2(b) is a flowchart showing a process for using the cervical seal device shown in FIGS. 4(a) and (b).

FIG. 3(a) shows an isometric view of a cartridge including a strap.

FIG. 3(b) shows a cross-sectional view of a cartridge including a strap.

FIG. 4(a) shows an embodiment of a cervical seal device including a squeeze handle.

FIG. 4(b) shows an distal end of a cervical seal device described in FIG. 4(a).

FIG. 5 shows an alternative embodiment of a cervical seal device.

FIG. 6(a) shows an enlarged view of a strap and mating tips of a cervical seal device.

FIG. 6(b) shows an alternative implementation of a strap.

FIG. 7(a) shows a strap having multiple capturing receptacles on one end.

FIG. 7(b) shows mating tips with alternative configurations.

FIG. 7(c) shows capturing receptacles with an alternative configuration.

FIG. 7(d) shows a strap having a ribbed surface.

FIG. 8(a) shows a front view of a strap including an offset.

FIG. 8(b) shows an isometric view of a strap including an offset and capturing receptacles.

FIG. 8(c) shows a plan view of a strap including an offset.

FIG. 8(d) is a flowchart of a process for using the cervical seal device shown in FIG. 5.

FIG. 9 shows an alternative embodiment of a cervical seal device.

FIGS. 10(a)-10(b) show clamping tips including a swivel feature.

FIG. 11(a) shows an alternative embodiment of a cervical seal device including a rotary handle.

FIG. 11(b) shows an enlarged view of a rotary handle including a control knob.

FIG. 11(c) shows an enlarged view of the connection between the arms and an outer shaft of an elongate member.

FIG. 11(d) shows an enlarged view of the connection between the arms and an inner shaft of an elongate member.

FIG. 12(a) shows a pair of detachable clamping tips.

FIG. 12(b) shows a clamping tip incorporating a detachment feature.

FIG. 12(c) is a flowchart of a process for using the cervical seal device shown in FIG. 11(a).

FIG. 13 shows an alternative embodiment of a cervical seal device.

Like reference symbols in the various drawings indicate like elements.

### DETAILED DESCRIPTION

In the following description, various implementations of the invention are described. However, it will be apparent to those skilled in the art that the implementations may be practiced with only some or all aspects of the invention. For purposes of explanation, specific numbers, materials and configurations are set forth in order to provide a thorough understanding of the implementations. However, it also will be apparent to one skilled in the art that the invention may be practiced without the specific details.

A device and technique for creating a seal between a cervical canal and a medical device positioned within the canal is described. The device includes a compressing member configured to exert a compressive force on a cervix, where the compressive force substantially creates a seal between the cervix and the medical device extending into the cervix. The device further includes an elongate member having a proximal end and a distal end. The compressing member is disposed at the distal end of the elongate member and the distal end is configured for insertion into a vaginal canal and into proximity of the cervix.

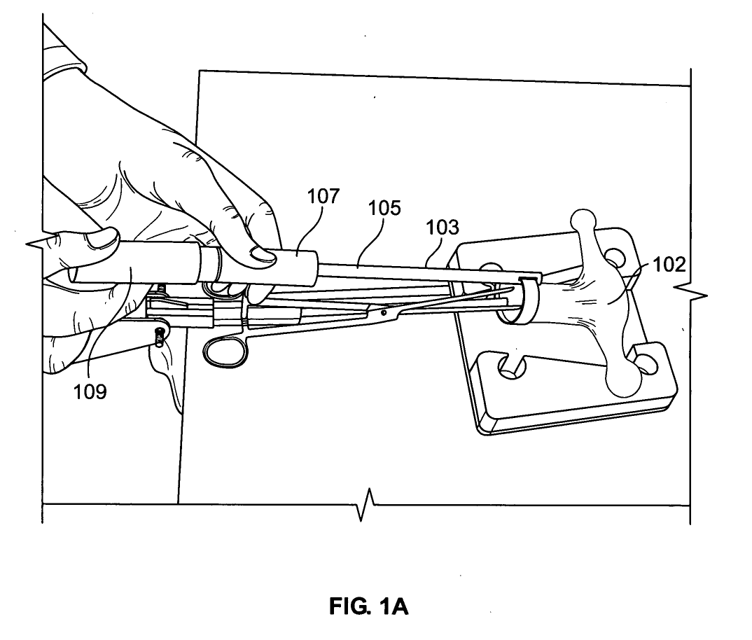

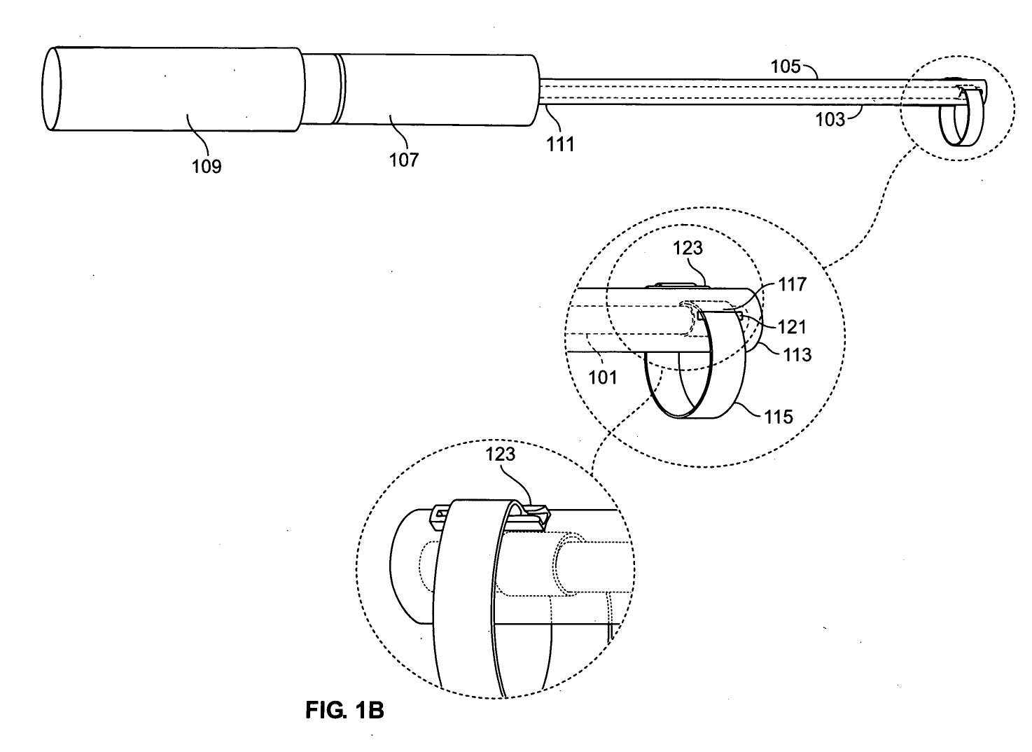

Referring to FIGS. 1(a) and 1(b), one implementation of a cervical seal device 100 is shown. In this implementation, the cervical seal device 100 employs a strap mechanism. For illustrative purposes, the cervical seal device 100 is shown in the context of an artificial cervix 102 to facilitate showing the features described herein. The cervical seal device includes an elongate member 105 having proximal and distal ends 111, 113. The elongate member is generally rigid axially. As shown, a compression force may be imparted onto the artificial cervix through the strapping mechanism provided at the distal end 113.

The cervical seal device includes an inner shaft 101 and an outer shaft 103. The inner shaft 101 and the outer shaft 103 may span the length of the elongate member 105. In one implementation, the outer shaft 103 is affixed to a handle portion 107 of a grip handle, while the inner shaft 101 is attached to a rotary portion 109 of the grip handle, so the inner shaft 101 can freely rotate inside the outer shaft 103. The handle can be attached to the elongate member 105 at or substantially near the proximal end 111.

A strap 115 is loaded at the distal end 113 of the elongate member 105. The strap 115 is captured at a first end 117 to the inner shaft 101 through an aperture 121 disposed on the outer shaft 103. At a second end 119 the strap 115 is slidingly received or traversely secured onto a holding element 123 affixed to the outer shaft 103 to generally form a loop. The strap 115 is not limited to the attachment configuration described above (e.g., both ends of the strap 115 can be attached to the inner shaft 101 or the outer shaft 103), and other attachment/engagement means also can be used. While the loop is shown with a generally circular shape, other curved shapes also can be established.

In one implementation, the cervical seal device 100 is disposable and made of injection molded thermoplastic and a malleable metal. Alternatively, the cervical seal device 100 can be reusable. In yet another alternative, the handle 107 and elongate member 105 can be reusable, and the strap 115 disposable. In another aspect, the elongate member 105 can optionally be molded to include a desired curvature for easing insertion of the cervical seal device 100 into the vaginal canal.

It should be understood that the functions of the cervical seal device as described above are not limited to operation related to a cervix, and may be utilized for other subject matters requiring a seal.

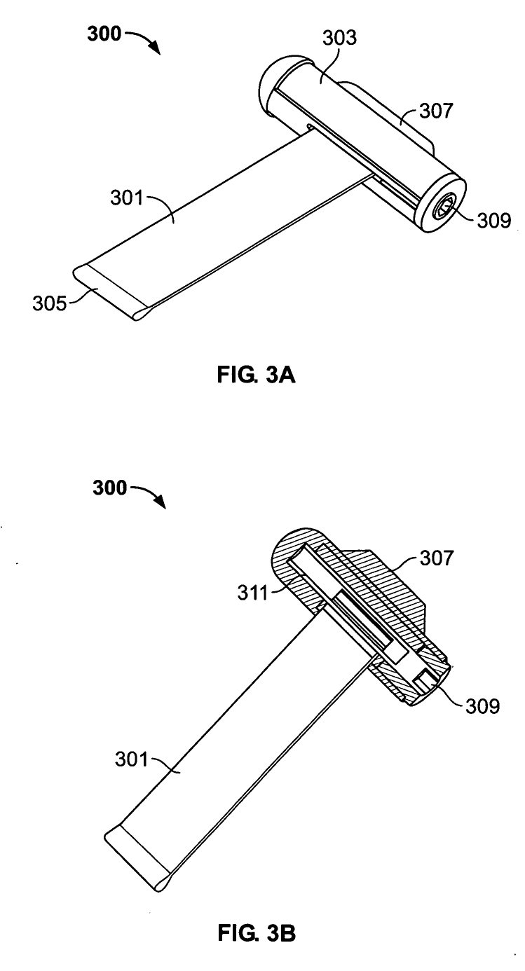

In yet another aspect, the strap is included in a cartridge that can be detachable from a distal end of the elongate member 105. FIG. 3(a) shows an isometric view of a cartridge 300 including a strap 301 therein. The strap 301 can be wound around an inner shaft and into or out of an outer shell 303 of the cartridge 300. While a first end of the strap may be adhered to the inner shaft or outer shell 303 of the cartridge 300, a second end 305 engages with or locks into a holding element, e.g., a clipping means or a slot provided on the outer shell 303 of the cartridge 300. In this implementation, the holding element 307 captures the second end 305 of the strap 301 to form a loop or other desired shape for positioning around a cervix. Further, the cartridge 300 includes a connector end 309, which mates with the distal end of the elongate member 105. The connector end 309 can be in the form of a snap-on coupling, pinning mechanism, threaded feature or any other suitable configuration for coupling the cartridge 300 to the elongate member 105.

FIG. 3(b) shows a cross sectional view of an implementation of the cartridge 300. In this implementation, the inner shaft 311 of the cartridge 300 is configured to freely rotate inside the outer shell 307 of the cartridge 300. The outer shell 307 mates with an elongate member to limit translational movement of the cartridge 300. In practice, a user may rotate a rotary portion 109 of a grip handle 107 (see FIG. 1(b)) connecting to the elongate member 105, causing the inner shaft 101 of the elongate member 105 to rotate. The inner shaft 101 engages the connector end 309 of the cartridge 300 thereby coupling to and rotating the inner shaft 311 of the cartridge 300. Rotation of the inner shaft 311 of the cartridge 300 winds or unwinds the strap 301 around the inner shaft 311, thereby lengthening or shortening the unwound portion of the strap 301. If the second end 305 of the strap is secured to the holding element 307, then winding or unwinding the strap 301 increases or decreases the radius of a loop formed by the secured strap 301.

In one implementation, the cartridge 300 is disposable while the elongate member 105 and the handle 107 are reusable. In another implementation, the cartridge 300 is reusable. Further, the strap 301 can be fabricated using elastic materials selected from, for example, silicone, natural rubber, viton, latex, Buena-N, E-PM rubber, urethane, Santoprene® (a registered trademark of Advanced Elastomer Systems LP of Akron, Ohio), and polyurethane. Alternatively, the strap 301 can be fabricated using non-elastic materials selected from, for example, PVC mesh, polyester mesh or filaments coated with PVC, nylon mesh, metal (e.g., stainless steel), mylar, polyetheretherketone (PEEK), polyimide, polypropelyne and acrylonitrile butadiene styrene (ABS).

In another implementation, the strap 301 can be injection molded, liquid molded, sewn from a flat sheet, riveted from flat stock or manufactured using other suitable manufacturing techniques for elastic or non-elastic materials. The strap 301 can also be molded to include features (e.g., bumps) suitable for enhancing friction on a cervix.

The grip handle 107 can be disposable or reusable. The handle 107 can be constructed using metal or plastic materials, such as stainless steel, PEEK, ultem, ABS and polycarbonate. For added strength and torque resistance, the handle 107 can optionally be molded into a composite structure. To provide added rigidity and resist torque, the composite structure may be a braid, including a base coat and a top coat.

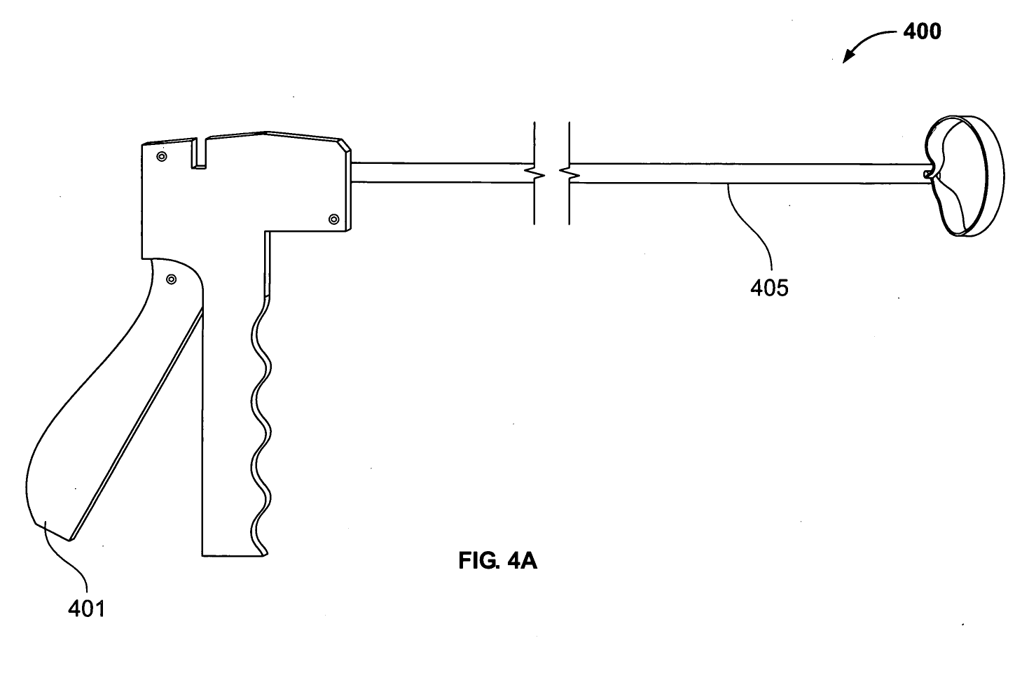

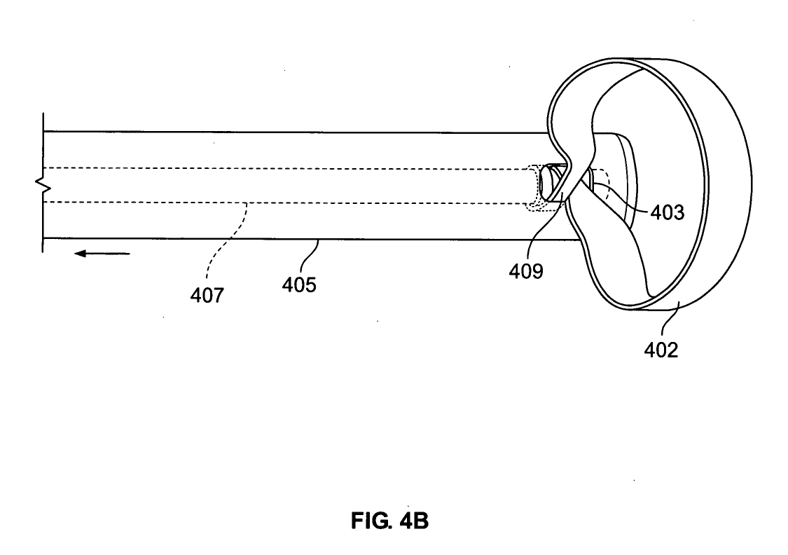

While a grip handle 107 has been described, other configurations of handles, for example, a squeeze handle may also be used. FIG. 4(a) shows an implementation of a cervical seal device 400 including an exemplary squeeze handle. FIG. 4(b) shows a distal end of the cervical seal device 400. Referring to FIG. 4(a) in conjunction with FIG. 4(b), the size of a loop formed by the strap 402 is controlled by a squeeze handle 401. The proximal end of the strap 409, attaches to an inner shaft 407. As a user squeezes the handle 401, the inner shaft 407 retracts within an outer sheath 405, pulling some of the strap 402 into an aperture 403 formed at the distal end of the outer sheath 405. The loop formed by the strap 402 thereby decreases in size. In operation, the strap 402 is first positioned around the cervix, and then handle 401 squeezed to reduce the loop size and thereby apply a compressive force to the cervix. In one implementation, the strap 402 can narrow (see element 409) in the vicinity of the strap 402 that retracts into the outer sheath 405. In another implementation a first end of the strap 402 can be affixed to the outer sheath 405 and only a second end of the strap is affixed to the inner shaft 407 and retracts into the aperture 403 upon squeezing the handle 401. In this implementation, although only the second end of the strap retracts, the loop size is still reduced and a compressive force can be applied to a cervix.

Other handle configurations also may be used, such as those employing mechanical gears and components for converting a squeeze motion into a rotary motion to allow the strap to wind and unwind around the inner shaft 311. In one implementation, a ratchet locking mechanism may be provided. If employed, a ratchet locking mechanism can help maintain or adjust the amount of winding or unwinding of the strap 301 from the inner shaft 311. In one aspect, increments or intervals of the ratchet locking mechanism can be chosen to accommodate a substantial range of about 0.5 mm to 5 mm of strap length per ratchet.

The cervical seal devices described herein can be used for any procedure where a seal around the cervix is desired. One example of such a procedure is performing a cavity assessment phase of an endometrial ablation procedure, as described above. However, other procedures are possible where the cervical seal device may be used, including, but not limited to, a diagnostic hysteroscopy, rollerball ablation and resecting loop procedures including endometrial ablation, hysteroscopic myomectromy or polypectomy.

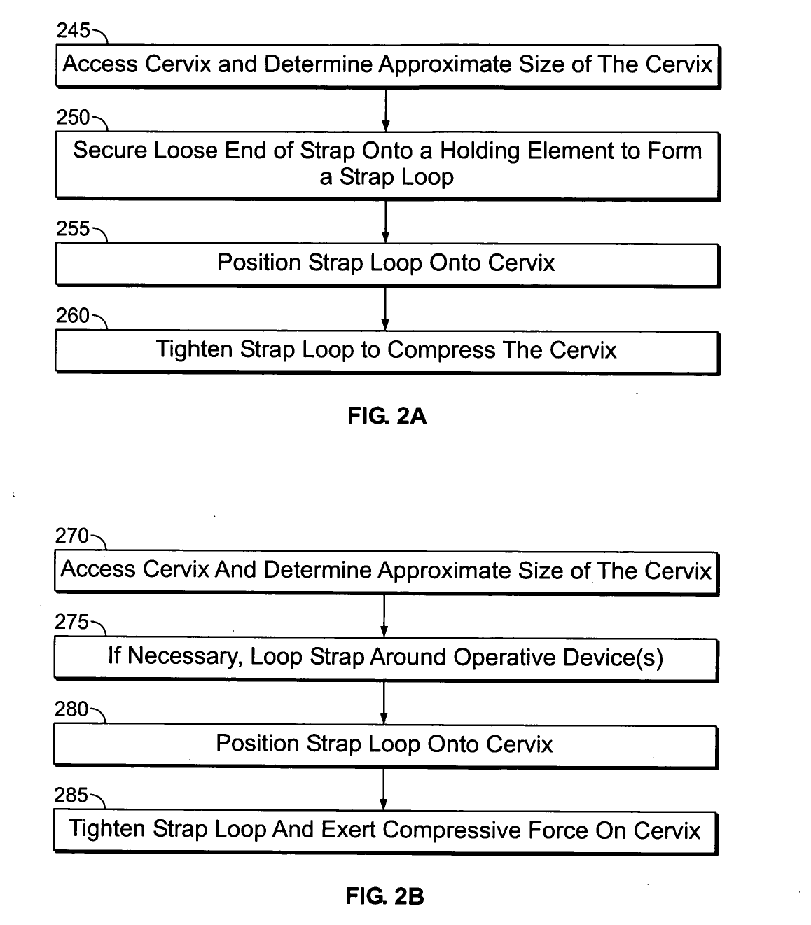

Referring to FIG. 2(a), a flowchart showing a process for using the cervical seal device 100 shown in FIGS. 1(a) and (b), which may or may not be used in combination with the cartridge shown in FIGS. 3(a) and (b) is shown. Optionally, the external cervical os is accessed (e.g., using a tenaculum) and, if possible, an approximate size of the cervix is determined (step 245). If there are one or more medical devices already positioned within the cervix (e.g., a speculum, electrode array device, or otherwise), the strap 301 is looped around the portions of the device external to the body and secured into the holding element 307 (step 250). If there are no such medical devices, the strap 301 can simply be looped and secured into the holding element 307. The distal end of the cervical seal device 100, including the secured strap, is inserted into the patient's body to the proximity of the external cervical os, and the strap 301 is positioned on the cervix (step 255). The strap 301 can be shortened, i.e., the strap wound to reduce the size of the loop formed by the strap 301, to thereby exert a compressive force on the cervix (step 260).

FIG. 2(b) is a flowchart showing a process for using the cervical seal device 400 shown in FIGS. 4(a) and (b). Optionally, the external cervical os is accessed (e.g., using a tenaculum) and, if possible, an approximate size of the cervix is determined (step 270). If there are one or more medical devices already positioned within the cervix (e.g., a speculum, electrode array device, or otherwise), the strap 402 is extended to loop around the portions of the device external to the body (step 275). If there are no such medical devices, the strap 402 can simply be extended to a loop size slightly larger then the anticipated size of the external cervix. The distal end of the cervical seal device 400, including the strap 402, is inserted into the patient's body to the proximity of the external cervical os, and the strap 402 is positioned on the cervix (step 280). The strap 402 can be shortened, i.e., the inner strap 407 contracted to reduce the size of the loop formed by the strap 402, to thereby exert a compressive force on the cervix (step 285).

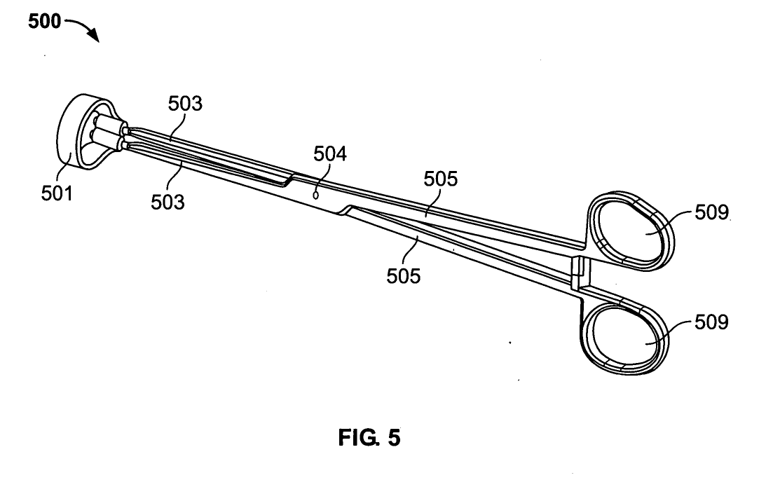

Referring now to FIG. 5, another implementation of a cervical seal device 500 is shown. In the implementation depicted, a strap 501 is mounted on cross arms 503 being pivotally and conventionally arranged to pivot about point 504 in a scissor-like motion. The proximal ends 505 of the cross arms 503 include corresponding handles 509, and the distal ends 507 incorporate mating tips for mating with capturing receptacles provided on the strap 501.

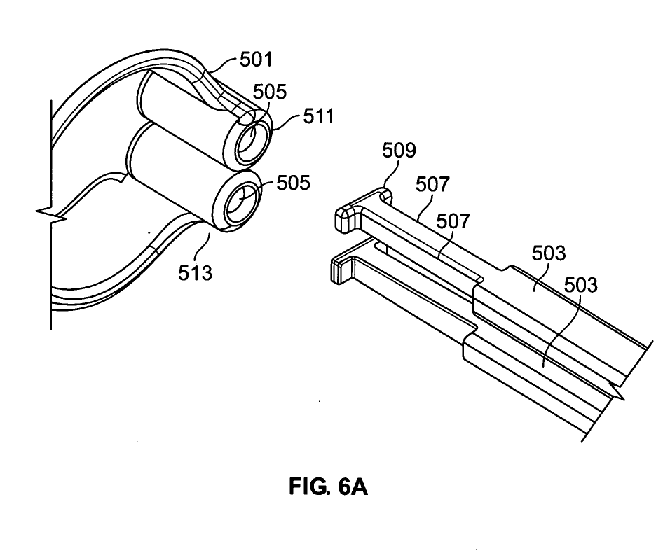

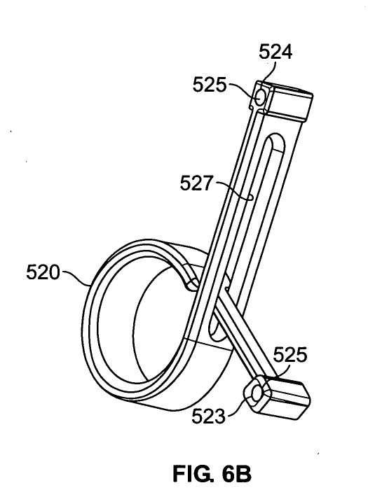

Referring to FIG. 6(a), an enlarged view of the strap 501 separated from the mating tips 509 is shown. The strap 501 includes a first capturing receptacle 505 at a first end 511 and a second capturing receptacle 505 at a second end 513. As the strap 501 is loaded onto the distal ends 507 of the arms 503, a user inserts the capturing receptacles 505 onto the mating tips 509 to ensure that the strap 501 does not dislodge when in use.

A ratchet locking mechanism for maintaining or adjusting the overall diameter of the strap 501 in discrete intervals may also be provided between the handles 509 if a handle 509 configuration as shown in FIG. 5 is employed. In one aspect, increments or intervals of the ratchet locking mechanism can be chosen to accommodate a substantial range of about 0.5 mm to 5 mm of spacing per ratchet.

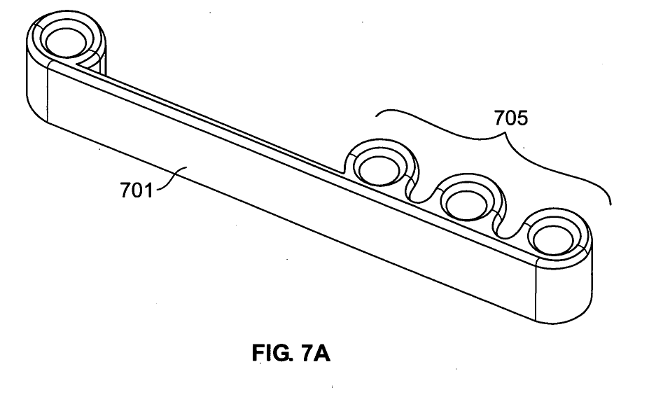

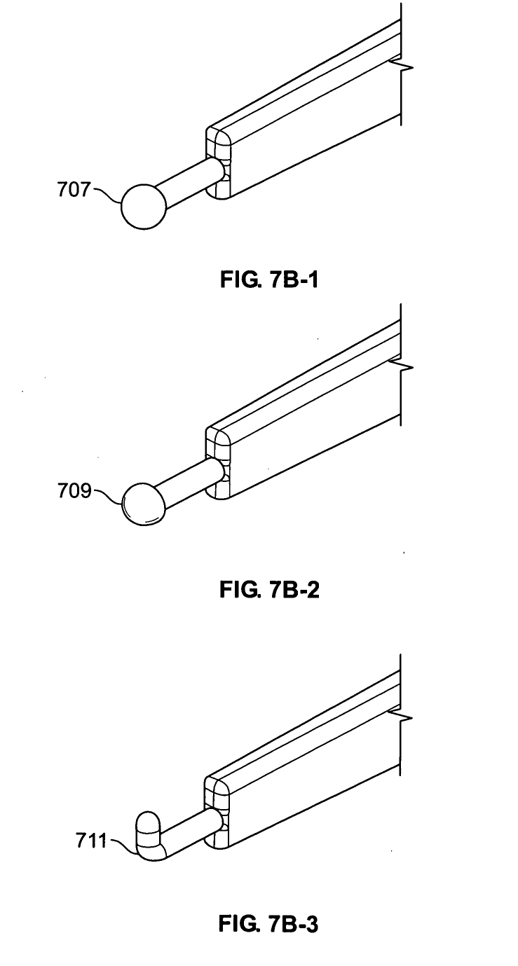

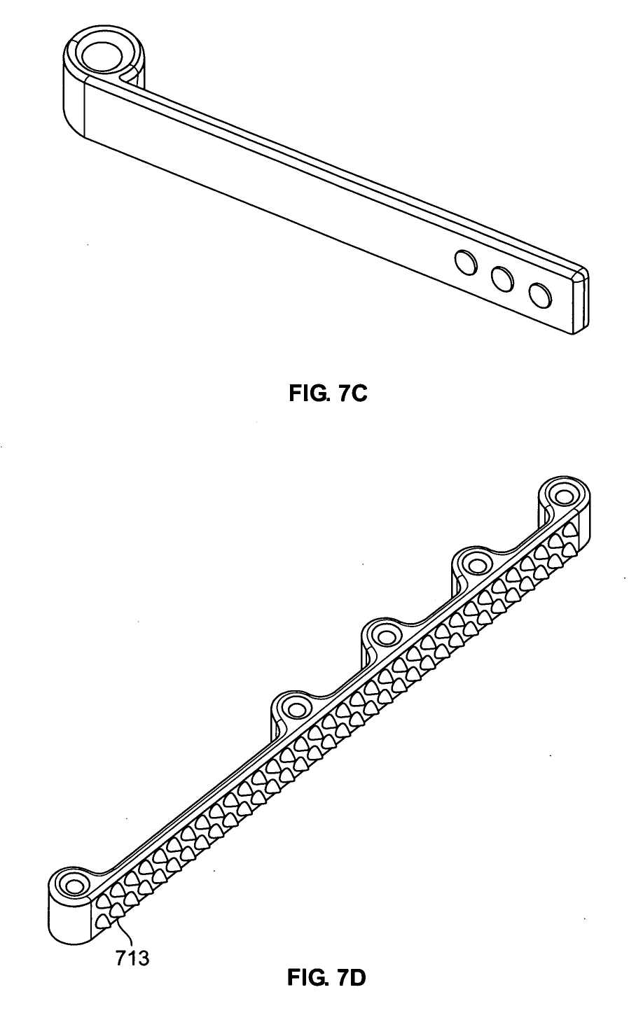

The strap 501 also may include more than two capturing receptacles to accommodate a wider range of outer cervix diameters. Referring to FIG. 7(a), a strap 701 is shown including a capturing receptacle 703 on a first end and multiple capturing receptacles 705 on a second end. In this implementation, a user can lengthen or shorten the strap length by selecting an appropriate capturing receptacle from the multiple capturing receptacles 705 on the second end, thereby accommodating different cervix sizes and/or allowing for different compressive forces to be applied to the cervix.

The shape or configuration of the mating tips is not limited to that shown (i.e., rectangular shape), and can be configured with other suitable design in the form of, for example, a ball tip 707, a hemispherical tip 709, a hook tip 711, a pan head or a side pin, as shown in FIG. 7(b). Similarly, the capturing receptacles are not limited to generally circular or oval shape, and can be replaced with holes (e.g., configured to mate with pins) as shown in FIG. 7(c), or other configurations suitable to mate with a mating tip. FIG. 7(d) shows a strap configured with a ribbed surface 713 for enhancing the hold and compression force imparted by the strap.

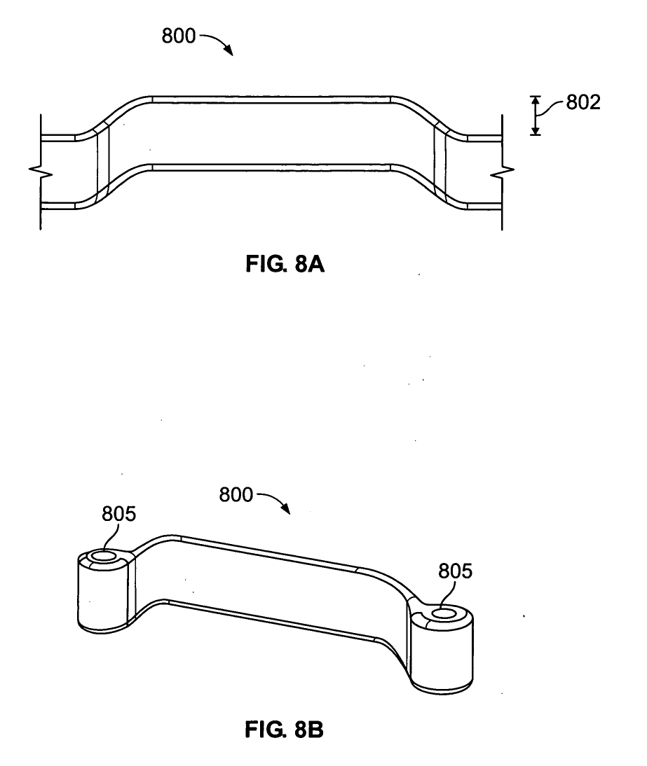

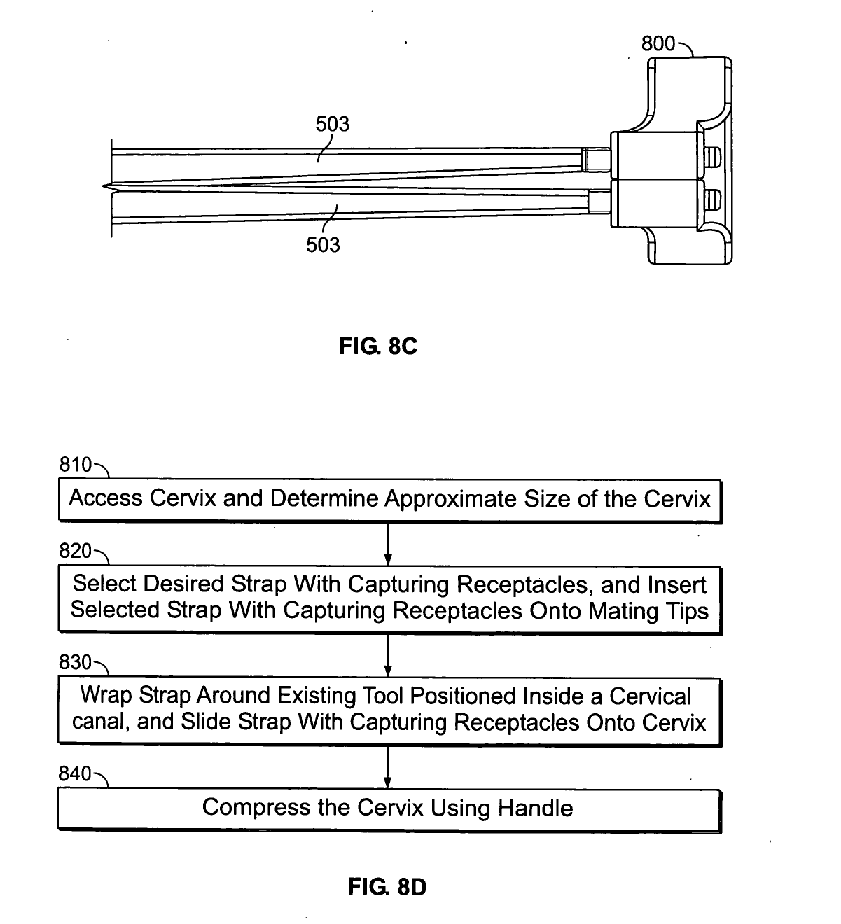

In one implementation, the strap is configured with a slight offset suitable for aligning with the mating tips 507 of the arms 503. FIG. 8(a) shows a front view of a strap 800 including an offset 802. FIG. 8(b) shows an isometric view of a strap 800 including an offset and capturing receptacles 805. FIG. 8(c) shows a plan view of the strap 800 aligned with the arms using an offset. In this implementation, the tips of the arms 503 are substantially flush with the strap 800.

In another implementation, the strap dimensions are approximately as follows: 62 mm to 125 mm in length, 2 mm to 20 mm in width and 0.25 mm to 4 mm in thickness, and can accommodate cervix diameters ranging from approximately 20 mm to 40 mm.

FIG. 8(d) is a flowchart demonstrating an exemplary process for using the cervical seal device 500 shown in FIG. 5. Optionally, a user accesses a cervix and determines the approximate size of the cervix (step 810). The user selects an appropriate strap length, or an appropriate capturing receptacle as in the case of a strap configured as shown in FIG. 7(a) (step 820). The user mates the capturing receptacles 505 of the strap 501 with mating tips 509 of the arms 503 of the cervical seal device 500 (step 830). Outside of the patient's body, the strap may be looped around any tool already positioned inside the cervical canal, and then slidingly inserted into the proximity of the cervix to position around the cervix. The first and second ends of the strap 501 are drawn together, for example, by squeezing the handles 509 of the cervical seal device 500 together. A compressive force is thereby applied to the cervix (step 840). If one or more medical devices are extending through the cervix, e.g., a speculum and/or tenaculum, a seal is created between the interior of the cervix and the one or more medical devices.

FIG. 6(b) shows an alternative implementation of a strap 520 that can be mated to mating tips 509 on the arms 503 shown in FIG. 5. In this implementation, spreading the arms 503 apart into an opened position has the effect of tightening the strap 520 (in contrast to the implementation discussed above that tightened the strap in the closed position). A first end 523 of the strap 520 is positioned through an aperture or slot 527 formed in a second end 524 of the strap 520. A capturing receptacle 525 is formed at each end of the strap 520 and is configured to mate with a mating tip 509 on a corresponding arm 503. In another implementation, two or more capturing receptacles 525 can be formed at either or both of the first and second ends 523, 524 of the strap 520.

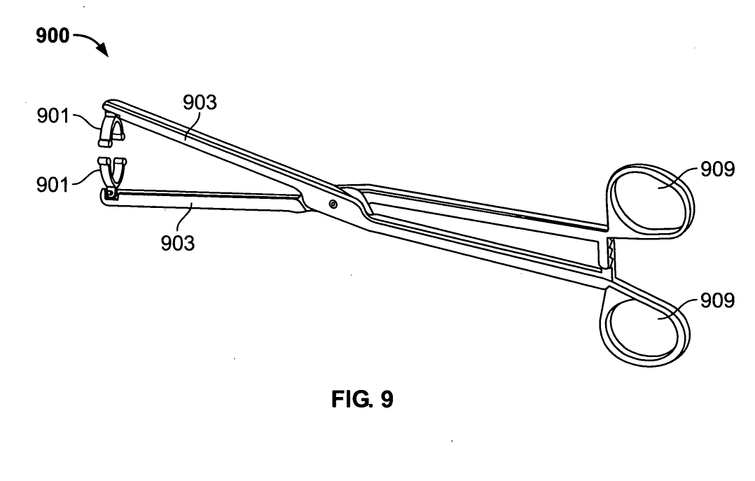

FIG. 9 illustrates another implementation of a cervical seal device. As depicted, the cervical seal device 900 includes two hemi-spherically shaped clamping tips 901 attached to arms 903 having handles 909 at their proximal ends. The arms 903 are pivotally connected to one another, and the handles 909 can be opened and closed in a scissor-like motion to move the distal ends of the arms 903, and thereby the clamping tips 901, between opened and closed positions. The clamping tips 901 are configured to approximately fit around cervixes of a range of diameters. Squeezing the handles 909 together once the clamping tips 901 are position around the exterior of a cervix applies a compressive force to the cervix.

In one implementation, the arms 903 can be integrated (e.g., injection molded) with the clamping tips 901. Alternatively, the clamping tips 901 can be detachable from the arms 903, which can provide a means by which to reuse the arms 903 and handle 909 while having disposable clamping tips 901.

Furthermore, because other surgical instruments (e.g., tenaculum and speculum) may be used in conjunction with a cervical seal device during a given surgical procedure, the cervical seal device may not always be positioned in-line with the cervix, potentially damaging neighboring tissues of the cervix and other vital organs by the clamping tips 901 or arms 903.

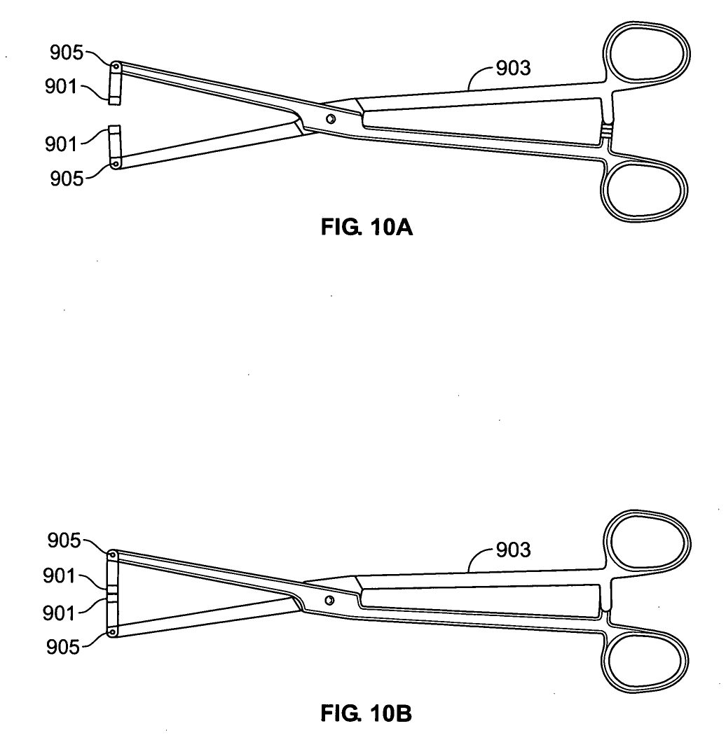

Accordingly, in one implementation, to strengthen cervix-tissue protection during insertion and retrieval of a cervical seal device, the clamping tips 901 include a swivel feature. Using this structure, the clamping tips 901 can pivot relative to the arms 903 about points 905. Referring to FIGS. 10(a) and (b), as the separation between the clamping tips (or handles 909) increases (FIG. 10(a)), the swivel feature allows the angle between each corresponding arm 903 and clamping tip 901 to be configured less acute. As the arms 903 are moved toward each other, the clamping tips 901 pivot about points 905 and the angle between each clamping tip 901 and corresponding arm 903 becomes less acute. This feature can allow the contacting surface of each clamping tip to remain substantially parallel to the exterior surface of the cervix and to each other, so as to prevent damage to neighboring tissue of the cervix. The contacting surface of the clamping tips also may be configured to remain substantially parallel to the exterior surface of the cervix regardless of the separation between the arms 903.

The clamping tips 901 cannot always be positioned inline with the cervix, as operative instruments positioned therein may occupy that position. In such cases, the arms 903 must exit the vagina at an angle to the cervix. The swivel features allows the clamping tips 901 to achieve a solid purchase on the cervix, even is at an angle to the cervix.

In another implementation, a ratchet locking mechanism can be provided to maintain or adjust a desired separation and spacing between the clamping tips 901 set by a user in discrete increments or intervals. The ratchet locking mechanism 912 permits the user to position the clamping tips 901 onto a cervix, and release a hold thereon without compromising a compressive force exerted on the cervix by the cervical seal device 900. In one aspect, increments or intervals of the ratchet locking mechanism can be chosen to accommodate a substantial range of about 0.5 mm to 5 mm of spacing per ratchet, and may support a diameter of about 50 mm of the enlarged geometry (i.e., in an opened position).

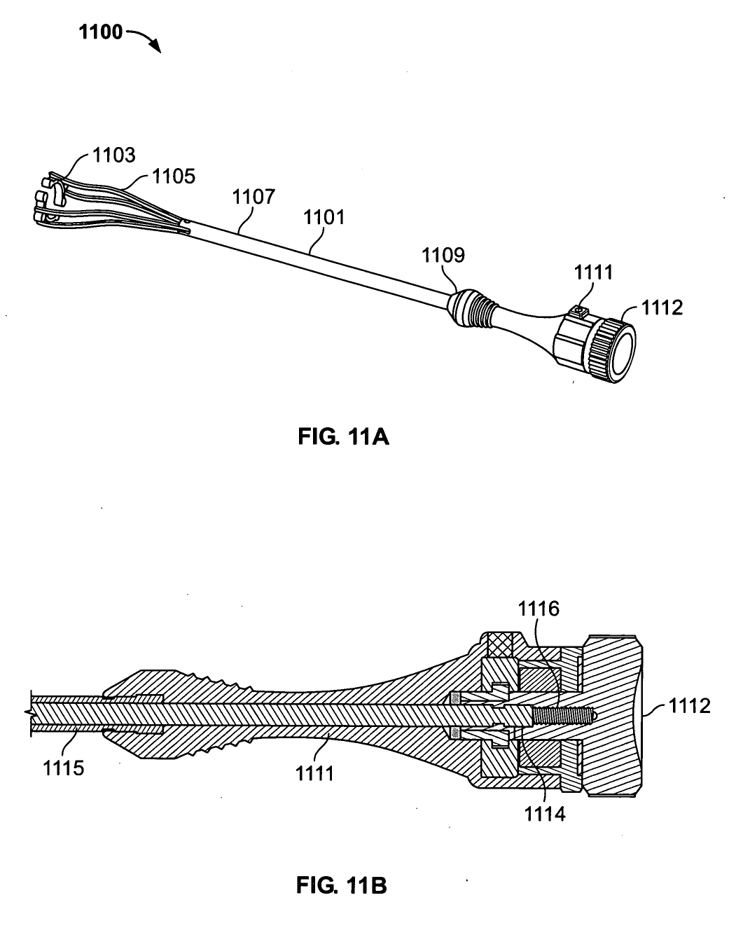

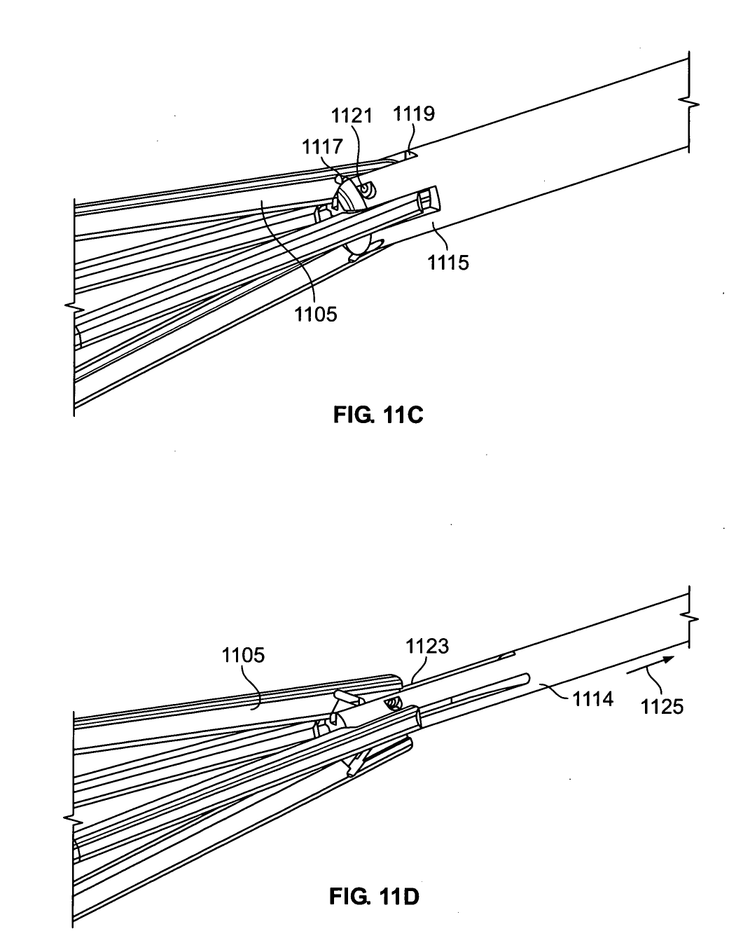

While an arm and finger-grip handle configuration has been described, other suitable devices can be used to position and manipulate the clamping tips 901. For example, an elongate member including a rotary handle having a control knob, may be employed to facilitate spacing of the clamping tips. One implementation employing a rotary handle is shown in FIG. 11(a). The cervical seal device 1100 includes a rotary handle 111 positioned at a proximal end of an elongate member 1101. A distal end 1107 of the elongate member 1101 is connected to clamping tips 1103 via the arms 1105.

Referring to FIG. 11(b), in one implementation, an inner shaft 1114 of the elongate member 1101 extends to couple with an internal mating thread 1116 of a control knob 1112 at the proximal end of the rotary handle 1111. As the control knob 1112 is rotated, for example, in clockwise direction, the inner shaft 1114 moves with reference to the outer shaft 1115 of the elongate member. The outer shaft 1115 is fixed to the handle 1111. Rotating the control knob 1112 causes the arms 1105 to deploy through angled slots 1119 via mating pins 1121 running across the outer shaft 1115 and each respective arm.

FIG. 11(c) illustrates an enlarged view of the connection between the arms 1105 and an outer shaft 1115 of the elongate member. As the arms 1105 pivot about mating pins 1121, they move relative to the outer shaft 1115 via angled slots 1119. FIG. 11(d) illustrates an enlarged view of the connection between arms 1105 and the inner shaft 1114 of the elongate member. Pins 1121 pivotally connect arms 1105 to inner shaft 1114. As the inner shaft 1114 axially translates in the direction of arrow 1125, the proximal ends of arms 1105 pivot about pins 1121 and the distal ends of arms 1105 are drawn radially inwardly and move toward a closed position as slots 1119 of arms 1105 translate along pins 1121. As the inner shaft 114 translates in the direction opposite arrow 1125, the distal ends of arms 1105 are moved radially outwardly (relative to the outer shaft 1115), and move toward an open position.

In one implementation, the cervical seal device is configured to be reusable or disposable. Where the cervical seal device is reusable, surgical stainless steel or other suitable medical grade hard plastic or composite material can be selected to fabricate the arms and/or clamping tips. The cervical seal device can be sterilized by methods including, but not limited to, autoclave, cold sterilant soak, hydrogen peroxide, Sterris, ETP, radiation or other suitable sterilization methods.

Alternatively, where the cervical seal device is disposable, the arms and/or clamping tips can be injection-molded using plastic materials selected from, for example, polyetheretherketone (PEEK), polysulfate, polypropelyne, acrylonitrile butadiene styrene (ABS) and ultem.

In one implementation, to reduce shaft deflection or arm bending stress caused by plastic materials, a metal member spanning the length of the arms can be provided to act as a non-deflectable beam to counter such deflection or bending stress. In this implementation, the arms can be injection molded over their corresponding metal member.

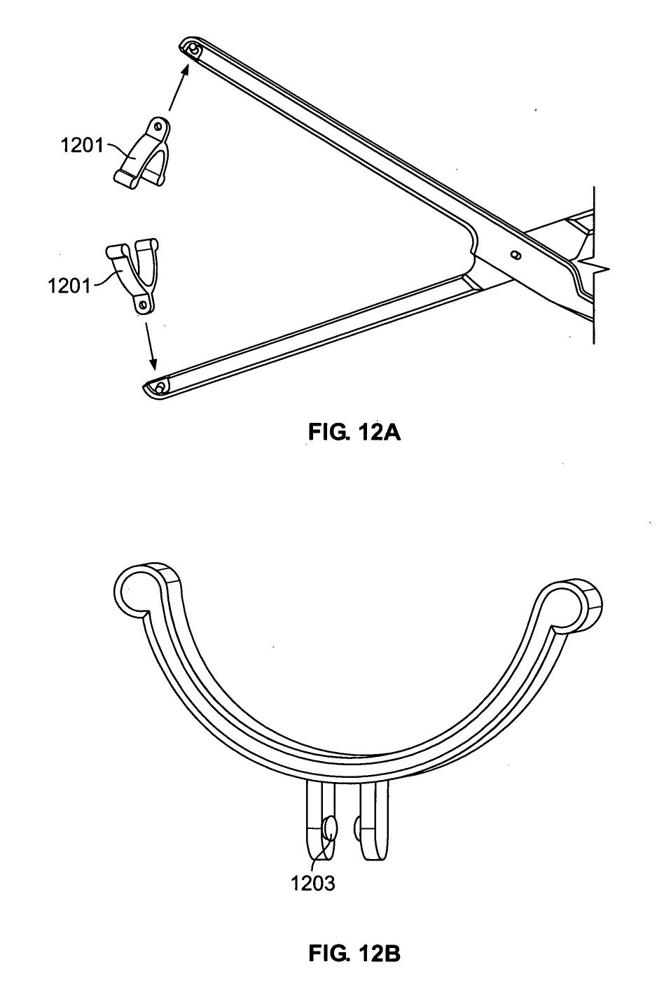

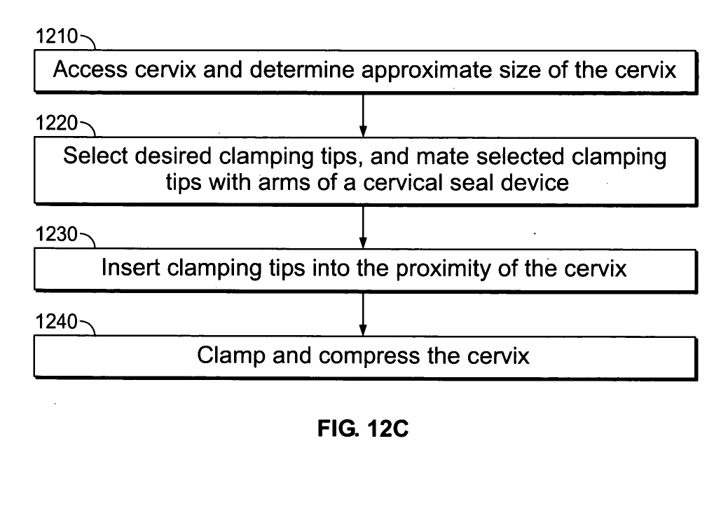

In another implementation, the clamping tips are detachable. Referring to FIG. 12(a), the hemi-spherical shaped clamping tips 1201 are detachable. In this implementation, the clamping tips 1201 can be disposed after a single use. The clamping tips 1201 can further be configured to include a snap-on feature to facilitate attachment and detachment of the clamping tips 1201. This is illustrated in FIG. 12(b), where each clamping tip can include a pair of protruding balls 1203 mating with a corresponding pair of receptacles (e.g., ball detents) disposed at the distal end of the arms. Other suitable coupling means for facilitating attachment and detachment of the compressing tips 1201 also can be employed.

Dimensions of the compressing tips 1201 can be configured to accommodate a wide range of cervical sizes. For example, a set of clamping tips with an effective inner diameter of about 20 mm can be designated for handling cervices having small diameters. Clamping tips with an effective diameter of about 50 mm can be supplied for sealing cervices having large diameters. An intermediate set of clamping tips with an effective inner diameter of about 30 mm also can be provided to manage cervices that are in an intermediate range.

While an atraumatic tip design (e.g., hemi-spherical) has been illustrated to prevent scraping cervical tissues (e.g., pinching or piercing) by edges of the clamping tips, one skilled in the art would appreciate that the clamping tips can be configured in any number of suitable shapes or degrees of curvature.

FIG. 12(c) is a flowchart demonstrating an exemplary method using a cervical seal device shown in FIG. 11(a). Optionally, a user may access a cervix and determine the size of the cervix (step 1210). The user selects appropriate clamping tips 1103, and mates the selected clamping tips 1103 with the arms 1105 of the cervical seal device 1100 (step 1230). The clamping tips 1103 attached to the arms 1105 of the cervical seal device are inserted into the proximity of the cervix, and the clamping tips 1103 are positioned about the exterior of the cervix. The control knob 1112 is rotated to move the clamping tips 1103 into a substantially closed position, so as to provide a compressive force on the cervix (step 1240).

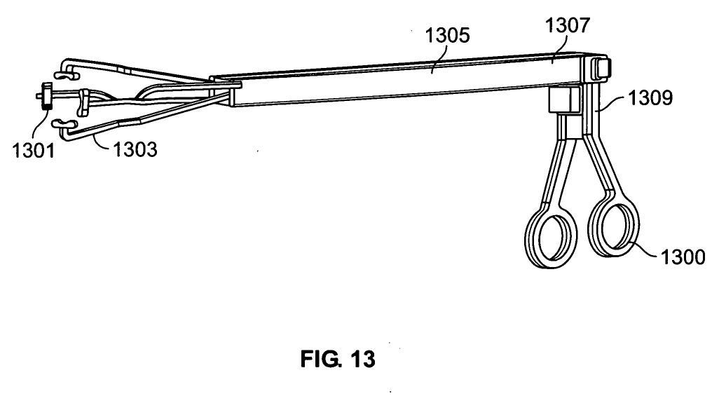

FIG. 13 illustrates another implementation of a cervical seal device. In the implementation depicted, the cervical seal device 1300 includes four clamping tips 1301 each of which is attached to the distal end of a corresponding arm 1303. The arms are attached to the distal end of an elongate member 1307. The elongate member 1307 includes an outer sheath 1305. A finger-grip handle 1309 is connected to a proximal end of the elongate member 1307.

When an “open position” is triggered by the finger grips of the handle 1309, the arms 1303 deploy radially outwardly to provide an enlarged geometry. In a “closed position”, the clamping tips 1301 moved toward each other, e.g., to clamp about the exterior surface of a cervix. In one implementation, the clamping tips 1301 can be configured to deploy substantially 90 degrees to the longitudinal axis of the elongate member 1307. The clamping tips 1301 can further be configured to be substantially parallel to the exterior surface of the cervix when deployed so as to prevent damaging the neighboring tissues of the cervix. In this configuration, the tips 1301 can pivot about the distal ends of arms 1303 so that the contacting surfaces of tips 1301 may remain substantially parallel with the exterior surface of the cervix independent of the angle between arms 1303 and the cervix. In another configuration, the tips 1301 may be shaped to match an exterior surface of a cervix (e.g., a curved inner surface and a radius of which matches that of the exterior surface of the cervix) to further reduce the likelihood of causing physical damage to neighboring tissues of the cervix.

Switching the clamping tips 1301 between the opened and the closed positions can be achieved in a similar manner as described above in reference to FIGS. 11(a) to (d). However, in the implementation shown in FIG. 13, a squeeze grip with handle 1309 is used to translate the outer sheath 1305 relative to an inner shaft to move the arms 1303, as compared to the rotary handle 1111 shown in FIG. 11(a). Squeezing the finger grips of the handle 1309 translates into a forward motion of the outer sheath 1305. As the outer sheath 1305 moves forward, the arms 1303 are drawn toward one another, moving the clamping tips 1301 into a closed position. As the finger grips are separated, the outer sheath 1305 retracts, and the clamping tips 1301 move into an opened position. Other configurations of a handle or mechanism for moving the arms 1303 can be used (for example, the rotary type handle shown in FIG. 11(a)), and the configuration shown is merely exemplary.

In general, those skilled in the art will recognize that the invention is not limited by the details described, instead, the invention can be practiced with modifications and alterations within the spirit and scope of the appended claims. The description is thus to be regarded as illustrative instead of restrictive on the invention.

A number of embodiments of the invention have been described. Nevertheless, it will be understood that various modifications may be made without departing from the spirit and scope of the invention. Accordingly, other embodiments are within the scope of the following claims. For example, the steps of the flowcharts shown in FIGS. 2(a), 2(b), 8(d) and 12(c) can be performed in a different order and still achieve desirable results.

What is claimed is:

1. A cervical seal device, comprising:
an elongate member having a distal end and a proximal end, the distal end including an inner element coaxially positioned inside an outer element; and
a strap including a first end and a second end, the first end connected to the inner element of the distal end of the elongate member and the second end connected to the outer element of the distal end of the elongate member, where the strap forms a loop having a radius that is variable by rotating the inner element of the distal end of the elongate member to wind or unwind a length of the strap;
where the distal end of the elongate member is configured for insertion into a vaginal canal to an exterior of a cervix and the loop formed by the strap is configured to position around the exterior of the cervix and apply a compressive force to the cervix.

2. The device of claim 1, where the compressive force applied to the cervix can be varied by rotating the inner element of the distal end of the elongate member to wind the strap and decrease the radius of the loop formed by the strap.

3. The device of claim 1, where the distal end of the elongate member comprises a detachable cartridge including the strap.

4. The device of claim 1, where the inner element of the distal end of the elongate member is adapted to rotate substantially freely inside the outer element.

5. The device of claim 1, where an unwound length of the strap is dependent upon a degree of rotation of the strap inside the inner element.

6. The device of claim 1, where the elongate member further comprises a rotary handle, the rotary handle adapted to rotate the inner element to adjust a radius of the loop formed by the strap.

7. The device of claim 1, further comprising a locking mechanism to maintain the radius of the loop.

8. The device of claim 1, where the strap is made of an elastic material selected from one of a group consisting of silicone, natural rubber, viton, latex, Buena-N, E-PM rubber, urethane, Santoprene, and polyurethane.

9. The device of claim 1, where the strap is made of a non-elastic material selected from one of a group consisting of PVC mesh, polyester mesh or filaments coated with PVC, nylon mesh, metal, mylar, polyetheretherketone (PEEK), polyimide, polypropelyne and acrylonitrile butadiene styrene (ABS).

10. The device of claim 1, where the outer element at the distal end of the elongate member is configured to vary the loop radius upon rotation of the outer element.

11. The device of claim 1, where the compressive force applied to the cervix is substantially circumferential.

12. The device of claim 1, where the compressive force applied to the cervix acts over a portion of the cervix.

13. The device of claim 1, where the strap includes a plurality of frictional elements formed thereon, the frictional elements configured to preserve a strap position on the cervix and maintain the compressive force applied to the cervix.

14. A cervical seal device comprising:
an outer sheath having a distal end and a proximal end and a lumen extending therebetween, the distal end including an aperture;
inner shaft positioned within the lumen of the outer sheath, a distal end of the inner shaft connected to a flexible member;
a flexible member having a first end and a second end, where the first end is connected to the inner shaft and the second end is connected to the outer sheath or to the inner shaft and where the flexible member forms a resizable loop extending from the aperture in the distal end of the outer sheath;
a handle attached to the proximal end of the outer sheath and the inner shaft, the handle including at least a first component configured to be moved relative to a second component in a squeezing motion, where squeezing the handle retracts the inner shaft and at least the first end of the flexible member connected thereto within the outer sheath thereby reducing a size of the loop formed by the flexible member;
wherein the distal end of the outer sheath is configured for insertion into a vaginal canal to an exterior of a cervix and the loop formed by the flexible member is configured to position around the exterior of the cervix and apply a compressive force to the cervix.

15. A cervical seal system, comprising:
a clamping device comprising:
two arms, each arm including a distal end and a proximal end with a handle positioned at the proximal end, where the two arms are pivotally attached to one another at a point between the distal and proximal ends such that the arms are pivotable relative to each other and where moving the proximal ends toward each other moves the distal ends toward each other from an opened position into a closed position;
a mating tip formed at the distal end of each arm and configured to mate with a capturing receptacle formed on a strap; and
a strap having at least two capturing receptacles mounted thereon, where each capturing receptacle is configured to receive a mating tip formed at the distal end of an arm, the strap thereby forming a loop configured to position around an exterior of a cervix and exert a compressive force on the cervix when the arms are moved into a substantially closed position.

16. The device of claim 15, where the strap includes more than two capturing receptacles to allow differing sized loops to be formed by the strap when mated with the mating tips.

17. The device of claim 15, further comprising a locking mechanism to maintain a radius of the loop formed by the strap.

18. The device of claim 15, where each mating tip is detachable from the corresponding arm.

19. The device of claim 15, where each arm is made of plastic materials selected from one of a group consisting of polyetheretherketone (PEEK), polysulfate, polypropelyne, acrylonitrile butadiene styrene (ABS) and ultem.

20. The device of claim 15, where each mating tip is made of plastic materials selected from one of a group consisting of polyetheretherketone (PEEK), polysulfate, polypropelyne, acrylonitrile butadiene styrene (ABS) and ultem.

21. The device of claim 15, where each arm includes a supporting member operable to reduce a shaft deflection or bending stress.

22. The device of claim 15, where each mating tip is one of a ball tip, hemispherical tip, pan head, hook tip or a side pin.

23. The device of claim 15, where the strap is made of an elastic material selected from one of a group consisting of silicone, natural rubber, viton, latex, Buena-N, E-PM rubber, urethane, Santoprene, and polyurethane.

24. The device of claim 15, where the strap is made of a non-elastic material selected from one of a group consisting of PVC mesh, polyester mesh or filaments coated with PVC, nylon mesh, metal, mylar, polyetheretherketone (PEEK), polyimide, polypropelyne and acrylonitrile butadiene styrene (ABS).

25. A method of providing a compressive force to a cervix, comprising:
securing a first end of a strap into a holding element formed at a distal end of an elongate member, where a second end of the strap is already secured to the distal end, the strap thereby forming an approximate loop;
inserting the elongate member through a vaginal canal into a proximity of the exterior of the cervix;
positioning the loop formed by the strap around the exterior of the cervix; and
winding a length of the strap into the distal end of the elongate member to decrease a size of the loop and thereby exert a compressive force on the cervix.

26. The method of claim 25, wherein inserting the elongate member includes wrapping the strap externally around an existing tool positioned inside the vaginal canal externally without retracting the existing tool prior to positioning the loop around the exterior of the cervix.

27. The method of claim 25, where the second end of the strap is secured to an inner element within the distal end of the elongate member and holding element is attached to an outer element of the distal end, and where winding a length of the strap comprises rotating a knob at a proximal end of the elongate member to rotate the inner element relative to the outer element.

28. The method of claim 25, where the second end of the strap is secured to an inner element within the distal end of the elongate member and the holding element is attached to an outer element of the distal end, and where winding a length of the strap comprises squeezing a handle at a proximal end of the elongate member to rotate the inner element relative to the outer element.

29. A method of providing a compressive force to a cervix, comprising:
inserting a distal end of an outer sheath having a distal end including an aperture, a proximal end, a lumen extending therebetween and an inner shaft positioned within the lumen into a vaginal canal to the vicinity of a cervix;
positioning a flexible member around the cervix, the flexible member having a first end and a second end, where the first end is connected to the inner shaft and the second end is connected to the outer sheath or to the inner shaft and where the flexible member forms a resizable loop extending from the aperture in the distal end of the outer sheath; and
squeezing a handle attached to the proximal end of the outer sheath and the inner shaft, the handle including at least a first component configured to be moved relative to a second component in a squeezing motion, where squeezing the handle retracts the inner shaft and at least the first end of the flexible member connected thereto within the outer sheath thereby reducing a size of the loop formed by the flexible member and providing a compressive force to the cervix.

30. A method of providing a compressive force to a cervix, comprising:
securing a first end of a strap onto a mating tip formed at a distal end of a first elongate arm and securing a second end of a strap onto a mating tip formed at a distal end of a second elongate arm, where the first and the second elongate arms are pivotally connected to one another at a point of rotation between distal and proximal ends of the arms, the strap thereby forming an approximate loop;
inserting the arms and the strap through a vaginal canal into a proximity of the exterior of the cervix;
positioning the loop formed by the strap around the exterior of the cervix; and
moving the distal ends of the arms toward one another to substantially close the loop formed by the strap and thereby exert a compressive force on the cervix.

31. The method of claim 30, where the strap includes at least two capturing receptacles on the second end of the strap, and securing the second end of the strap onto a mating tip comprises selecting a capturing receptacle on the second of the strap corresponding to a desired loop size.

32. A medical device for providing a compressive force to a cervix, the device comprising:
a substantially cylindrically shaped outer element;
an inner shaft positioned with the outer element; and
a strap including a first end and a second end, the first end connected to the inner element and the second end connected to the outer element, where the strap forms a loop having a radius that is variable by rotating the inner element to wind or unwind a length of the strap;
where the outer element and inner shaft are configured to connect to an elongate member configured for insertion into a vaginal canal to an exterior of a cervix and the loop formed by the strap is configured to position around the exterior of the cervix and apply a compressive force to the cervix as the inner element is rotated by an adjustment means included on the elongate member to reduce the radius of the loop formed by the strap.

33. The device of claim 32, where the strap is made of an elastic material selected from one of a group consisting of silicone, natural rubber, viton, latex, Buena-N, E-PM rubber, urethane, Santoprene, and polyurethane.

34. The device of claim 32, where the strap is made of a non-elastic material selected from one of a group consisting of PVC mesh, polyester mesh or filaments coated with PVC, nylon mesh, metal, mylar, polyetheretherketone (PEEK), polyimide, polypropelyne and acrylonitrile butadiene styrene (ABS).
